(19) United States
(12) Patent Application Publication
(10) Pub. No.: US 2026/0321344 A1
(43) Pub. Date: Oct. 15, 2026

(54) DETECTING TANDEM REPEATS AND DETERMINING COPY NUMBERS THEREOF

(71) Applicant: ILLUMINA, INC., San Diego, CA (US)

(72) Inventors: Marzieh Eslami Rasekh, Carlsbad, CA (US); Jeffrey Yuan, Long Island City, NY (US)

(21) Appl. No.: 19/482,665

(22) PCT Filed: May 23, 2024
(86) PCT No.: PCT/US2024/030761

§ 371 Date: Nov. 7, 2025

Related U.S. Application Data
(60) Provisional application No. 63/505,371, filed on May 31, 2023.

(51) Int. Cl. G16B 20/10; G16B 40/20

(52) U.S. Cl. CPC G16B 20/10; G16B 40/20

(57) ABSTRACT

In one aspect, the disclosed technology relates to systems and methods for estimating a copy number of repeat units in a target genomic region in a nucleic acid sample from a subject. In some embodiments, a machine learning model is trained to learn the relationship between the known copy numbers of repeat units in predetermined invariant tandem repeat loci and specific features of the predetermined loci and specific features of training genomic regions in the nucleic acid sample from the subject that correspond to the predetermined loci. The copy number of repeat units in the target genomic region can be estimated by applying the trained machine learning model to specific features of a tandem repeat locus corresponding to the target genomic region and specific features of the target genomic region. In another aspect, the disclosed technology relates to computer-readable media including instructions for performing the disclosed methods.

### CROSS-REFERENCE TO RELATED APPLICATIONS

This application claims priority to U.S. Provisional Application No. 63/505,371, filed May 31, 2023, the content of which is incorporated by reference in its entirety.

### BACKGROUND

### Field

The disclosed technology relates to the field of nucleic acid sequencing. More particularly, the disclosed technology relates to detecting and identifying tandem repeats (TRs)—for example variable number tandem repeats (VNTRs)—in a sample nucleic acid and determining the copy numbers of the TRs.

### Description of the Related Art

Accurate detection of TRs has long been complicated by the low-complexity nature of TR regions. In some cases, the large size of the repetitive sequences in TRs further complicates the detection process. There exists a continuing need for improving the detection and characterization of TRs in nucleic acid sequencing technologies, especially since VNTRs account for a significant proportion of between-genome variations in humans.

### SUMMARY

In one aspect, the disclosed technology relates to a method of estimating a copy number of repeat units in a target genomic region in a nucleic acid sample from a subject, the target genomic region corresponding to a tandem repeat locus, the method comprising: obtaining known copy numbers of repeat units in predetermined invariant tandem repeat loci; training a machine learning model to learn the relationship between: the obtained copy numbers of repeat units in the predetermined invariant tandem repeat loci, and specific features of the predetermined loci and specific features of training genomic regions in the nucleic acid sample from the subject that correspond to the predetermined loci; obtaining paired-end sequence reads of the target genomic region, and estimating the copy number of repeat units in the target genomic region by applying the trained machine learning model to specific features of the tandem repeat locus and specific features of the target genomic region.

In some embodiments, the specific features of the tandem repeat locus comprise: a size of the tandem repeat locus, a size of a repeat unit in the tandem repeat locus, or a GC content of the tandem repeat locus.

In some embodiments, the specific features of the target genomic region comprise: a number of paired-end sequence reads resulting from the target genomic region that are spanning the tandem repeat locus, a number of paired-end sequence reads resulting from the target genomic region that are overlapping the tandem repeat locus, a number of paired-end sequence reads resulting from the target genomic region that are overlapping the start and end positions of the tandem repeat locus, or a number of paired-end sequence reads resulting from the target genomic region that are contained inside the tandem repeat locus.

In some embodiments, obtaining the paired-end sequence reads of the target genomic region comprises: performing paired-end sequencing of the nucleic acid sample from the subject, aligning the paired-end sequence reads to a reference genomic sequence for the species of the subject, and obtaining the paired-end sequence reads that align to the tandem repeat locus on the reference genomic sequence.

In some embodiments, the known copy numbers of repeat units in the predetermined invariant tandem repeat loci are obtained from a database or a reference genomic sequence.

In some embodiments, each of the predetermined loci is invariant among subjects of the same species.

In some embodiments, the machine learning model is a XGBoost model, a regression model, a random forest model or a decision tree model.

In some embodiments, training the machine learning model comprises: inputting the obtained copy numbers of repeat units in the predetermined invariant tandem repeat loci, the specific features of the predetermined loci, and the specific features of the training genomic regions corresponding to the predetermined loci to a machine learning module; optimizing the machine learning model using the machine learning module and said inputs; and outputting the trained machine learning model wherein the model parameters characterizing the relationship between the obtained copy numbers and the specific features of the predetermined loci and the specific features of the training genomic regions are optimized. In some embodiments, optimizing the machine learning model comprises: estimating predicted copy numbers of repeat units in the predetermined invariant tandem repeat loci by applying the machine learning model to the specific features of the predetermined loci and the specific features of the training genomic regions corresponding to the predetermined loci; comparing said predicted copy numbers with the obtained copy numbers of repeat units in the predetermined invariant tandem repeat loci; and updating the machine learning model based in part on said comparison.

In some embodiments, the specific features of the predetermined loci comprise: a size of the predetermined loci, a size of a repeat unit in the predetermined loci, or a GC content of the predetermined loci.

In some embodiments, the specific features of the training genomic regions corresponding to the predetermined loci comprise: a number of paired-end sequence reads resulting from the training genomic regions that are spanning the predetermined loci, a number of paired-end sequence reads resulting from the training genomic regions that are overlapping the predetermined loci, a number of paired-end sequence reads resulting from the training genomic regions that are overlapping the start and end positions of the predetermined loci, or a number of paired-end sequence reads resulting from the training genomic regions that are contained inside the predetermined loci.

In some embodiments, the disclosed method further comprises reporting a confidence level for said estimation, wherein the confidence level is determined based in part on control genomic regions in the nucleic acid sample that correspond to control loci comprising invariant tandem repeats. In some embodiments, each of the control loci is invariant among subjects of the same species. In some embodiments, determining the confidence level comprises: estimating predicted copy numbers of repeat units in the invariant tandem repeats of the control loci by applying the machine learning model to specific features of the control loci and specific features of the control genomic regions; comparing said predicted copy numbers with known copy numbers of repeat units in the invariant tandem repeats of the control loci; and determining the confidence level based on said comparison. In some embodiments, the known copy numbers of repeat units in the invariant tandem repeats of the control loci are obtained from a database or a reference genomic sequence. In some embodiments, the specific features of the control loci comprise: a size of the control loci, a size of a repeat unit in the control loci, or a GC content of the control loci. In some embodiments, the specific features of the control genomic regions comprise: a number of paired-end sequence reads resulting from the control genomic regions that are spanning the control loci, a number of paired-end sequence reads resulting from the control genomic regions that are overlapping the control loci, a number of paired-end sequence reads resulting from the control genomic regions that are overlapping the start and end positions of the control loci, or a number of paired-end sequence reads resulting from the control genomic regions that are contained inside the control loci.

In some embodiments, the nucleic acid sample comprises both paternal and maternal nucleic acids for the subject.

In some embodiments, the nucleic acid sample is extracted from cells, a cell-free DNA sample, an amniotic fluid, a blood sample, a biopsy sample, or any combination thereof, of the subject.

In some embodiments, the subject is a human.

In some embodiments, the tandem repeat locus is a variable number tandem repeat (VNTR) locus.

In some embodiments, a repeat unit in the tandem repeat locus is longer than about 700 base pairs in length.

In some embodiments, the tandem repeat locus is part of a macrosatellite or a minisatellite.

In some embodiments, each paired-end sequence read is about 100 base pairs to about 500 base pairs in length.

In some embodiments, paired-end sequence reads for the nucleic acid sample is generated by whole genome sequencing (WGS).

In some embodiments, the paired-end sequence reads for the nucleic acid sample is generated by a next generation sequencing reaction.

In some embodiments, each paired-end sequence read is obtained from a nucleic acid cluster on a solid substrate.

In some embodiments, the nucleic acid cluster on the solid substrate is generated by a bridge amplification process.

In another aspect, the disclosed technology relates to a system for estimating a copy number of repeat units in a target genomic region in a nucleic acid sample from a subject, the system comprising: a nucleic acid sequencer; non-transitory memory configured to store executable instructions; and a hardware processor in communication with the nucleic acid sequencer and the non-transitory memory, the hardware processor programmed by the executable instructions to perform the methods disclosed herein. In some embodiments, the hardware processor is configured to receive paired-end sequence reads from the nucleic acid sequencer. In some embodiments, the hardware processor is configured to control the nucleic acid sequencer to perform sequencing of the nucleic acid sample. In some embodiments, the hardware processor is configured to output, on a display, the estimated copy number of repeat units in the target genomic region.

In yet another aspect, the disclosed technology relates to a computer-readable medium comprising instructions that when executed perform a method of estimating a copy number of repeat units in a target genomic region in a nucleic acid sample from a subject, the target genomic region corresponding to a tandem repeat locus, the method comprising: obtaining known copy numbers of repeat units in predetermined invariant tandem repeat loci; training a machine learning model to learn the relationship between the obtained copy numbers of repeat units in the predetermined invariant tandem repeat loci and specific features of the predetermined loci and specific features of training genomic regions in the nucleic acid sample from the subject that correspond to the predetermined loci; obtaining paired-end sequence reads of the target genomic region; and estimating the copy number of repeat units in the target genomic region by applying the trained machine learning model to specific features of the tandem repeat locus and specific features of the target genomic region.

### BRIEF DESCRIPTION OF THE DRAWINGS

Features of examples of the present disclosure will become apparent by reference to the following detailed description and drawings, in which like reference numerals correspond to similar, though perhaps not identical, components. For the sake of brevity, reference numerals or features having a previously described function may or may not be described in connection with other drawings in which they appear.

FIG. 1A and FIG. 1B show a non-limiting exemplary illustration of a VNTR in a reference sequence. FIG. 1A shows the sequence of the repeat unit (SEQ ID NO: 1). FIG. 1B shows an alignment between read pairs within multiple repeat units.

FIG. 2 shows a non-limiting exemplary illustration of a VNTR in a reference sequence, with a repeat unit layout between different copy number variants of human subjects from different countries.

FIG. 3 schematically illustrates an example process involving using read depth information to predict the total copy number of repeat units in a TR in a sample according to some embodiments of the disclosed technology.

FIG. 4 is a block diagram that schematically illustrates an example machine learning module for detecting and identifying TRs according to some embodiments of the disclosed technology.

FIG. 5 is a flow chart that schematically illustrates an example method of estimating a copy number of repeat units in a target genomic region in a sample according to some embodiments of the disclosed technology.

FIG. 6A is a block diagram of an exemplary sequencing system that may be used to perform the disclosed methods.

FIG. 6B is a block diagram of an exemplary computing device that may be used in connection with the exemplary sequencing system of FIG. 6A.

### DETAILED DESCRIPTION

All patents, patent applications, and other publications, including all sequences disclosed within these references, referred to herein are expressly incorporated herein by reference, to the same extent as if each individual publication, patent or patent application was specifically and individually indicated to be incorporated by reference. All documents cited are, in relevant part, incorporated herein by reference in their entireties for the purposes indicated by the context of their citation herein. However, the citation of any document is not to be construed as an admission that it is prior art with respect to the present disclosure.

### Overview

Tandem repeats (TRs) are regions in the genome which have repetitions of a “pattern sequence” (or a “repeat unit”). Variable number tandem repeats (VNTRs) are TRs that may differ in the number (“copy number”) of repeat units across the population. The changes in the copy numbers of TRs in the human genome have been linked to gene silencing, differences in gene expression, genetic variations and various diseases. Large TRs have been linked to various phenotypes and diseases, such as susceptibility to human type 1 diabetes, chronic obstructive pulmonary disease (COPD), epilepsy, Parkinson disease, etc. However, very few large TRs (e.g., those having an array size larger than 1000 bp) have been studied in large cohorts due to the lack of reliable and efficient tools in the literature.

While methods exist for detecting, identifying or estimating the copy numbers of smaller TRs (e.g., those having an array size of less than a few hundred base pairs) in the human genome in conjunction with sequencing by synthesis (SBS) technologies, reliable and efficient methods for detecting, identifying or estimating larger TRs (e.g., those that are hundreds of base pairs or longer) are not readily available in short-read SBS technologies. The lack of a reliable and efficient method for detecting, identifying or estimating larger TRs in short-read SBS technologies is mainly due to the difficulty of performing secondary analysis (i.e., alignment and/or assembly of nucleic acid fragments/reads, and/or determination of genetic variants) on tandem repeats. On the one hand, short SBS reads cannot span large TRs; on the other hand, the repetitive nature of the TRs causes unreliable alignments of the SBS reads in the TR regions.

In the existing literature, the detection of copy number changes of large TRs usually falls in the domain of copy number variation (CNV) calling. However, current CNV tools are not able to distinguish TR patterns and positions, and they cannot detect sequence variations at the resolution of single repeat unit deletions/insertions, as they are generally designed for larger genomic regions. Moreover, current CNV tools are not designed to determine copy number information specific to TR regions, and generally only determine copy number changes over a fixed or customized window of the genome. In addition, in existing CNV tools, the read depth of the TR regions is set to be the same as the default read depth of the whole genome, without properly taking into account the repetitive nature of TRs and the lack of sufficient high-quality alignments in the TR regions. Therefore, existing CNV tools may misrepresent the copy number of the TR regions.

In some aspects, disclosed herein are systems and methods for detecting, identifying, determining or estimating the total copy numbers (or changes thereof) of larger TRs by using short-read SBS technology in a reliable and high throughput manner. The disclosed methods can perform secondary analysis in a way that can properly take into account the repetitive nature of TRs. In some embodiments, the disclosed methods specifically examine the “TR catalog”, which include certain pre-selected target genomic regions that correspond to tandem repeat loci in the reference genome, in contrast to current CNV tools that generally only examine a fixed or customized window of the genome. Moreover, the disclosed methods may utilize information from pre-determined loci of tandem repeats as a standard for calibration. In some embodiments, the disclosed methods may be applied in conjunction with whole genome sequencing of human genomes. Examples of the larger TRs of interest may include mini-satellites and macro-satellites, which are sometimes defined as TRs having repeat units of size larger than about 10 bp and about 100 bp, respectively. In some cases, mini-satellites may be defined as TRs having repeat units of size larger than about 5 bp, 6 bp, 7 bp, 8 bp, 9 bp, 15 bp, 20 bp, etc. In some cases, macro-satellites may be defined as TRs having repeat units of size larger than about 50 bp, 60 bp, 70 bp, 80 bp, 90 bp, 150 bp, 200 bp, etc.

Briefly, in some aspects of the disclosed methods, after aligning the SBS reads to the reference genome (e.g., a reference human genome), a machine learning (ML) model is used to learn the relationship between the total copy numbers of TRs (which would include both paternal and maternal copies in the case of a diploid organism, for example) and various features, including some TR sequence features and some sample-specific features or sequencing-read features. In some embodiments, the disclosed methods may take as input a set of predefined invariant TR regions (i.e., highly conserved regions with approximately fixed copy numbers of repeat units across a large cohort of population samples) and a set of TR regions of interest with unknown copy numbers of repeat units (referred to as the TR catalog). In some embodiments, the invariant TR regions are used to train a ML model in an “online”/sample- or dataset-specific manner, and the trained ML model is then used to predict/estimate the copy numbers of the TR catalog.

In some embodiments of the disclosed methods, training of the ML model may occur in parallel with or in real time during secondary analysis of the sequencing reads of the individual sample of interest. In alternative or additional embodiments, training of the ML model may occur before alignment of all the sequencing reads of the individual sample of interest is complete; for example, the disclosed methods may be performed to analyze certain genomic regions in parallel with the sequence alignment processes of other genomic regions. In alternative or additional embodiments, training of the ML model may occur after alignment of all the sequencing reads of the individual sample of interest is complete; for example, the disclosed methods may be performed on BAM files. In alternative or additional embodiments, training of the ML model and/or secondary analysis of the sequencing reads may overlap with or occur while the sequencer is still generating sequencing reads (i.e., before sequencing of the individual sample is complete); for example, the disclosed methods may be performed in parallel with the sequencing process (e.g., in an on-instrument analysis). In alternative or additional embodiments, training of the ML model and/or secondary analysis of the sequencing reads may occur after sequencing of the individual sample is complete.

While some ML approaches have been used in certain existing methods to analyze SBS sequencing reads, training of the ML model in such existing methods is not performed in a sample- or dataset-specific manner. Such existing methods learn the parameters of the ML model “offline” from a cohort of pre-existing datasets without taking into account the individual sample of interest (and usually prior to sequencing the individual sample of interest or prior to analyzing the dataset of interest/sequencing reads from the individual sample), and then apply the pretrained ML model on the dataset of interest/sequencing reads from the individual sample. Moreover, while some ML approaches have been applied to analyze specific genomic loci/genes that are large TRs, existing methods have not applied ML approaches to analyze VNTR regions in a genome-wide scale in the ways disclosed herein.

### Variable Number Tandem Repeats

Variable number tandem repeats (VNTRs) are a class of structural variants that include tandem repeats of patterns, for example patterns larger than 10 base pairs (bps), and that differ in copy number among the genomes of individuals of a species. While VNTRs cover <5% of the human genome, about 50% of all structural variants (variants greater than 50 bp) are VNTRs. In some cases, a VNTR can have fewer than 20% mismatches for an exact repeat. In some cases, VNTRs can have small variants, such as SNPs and indels in the repetitive sequences. On average one person has about 2.2 mega base pairs (Mbps) of deleted sequence and about 5.7 Mbps of inserted sequence in VNTRs. Variations in VNTRs can depend on the populations within a species.

Some VNTRs are known to be associated with genetic diseases, such as bipolar disorder, MCKD1, stroke, CAD, FSHD, ADHD, Parkinson's, diffuse panbronchiolitis (DPB), monogenic diabetes, TID, T2D, obesity, OCD, osteochondritis dissecans, Kawasaki, ATF in stroke, BPSD, Alzheimer's, anxiety, schizophrenia, metastatic colorectal cancer, Kawasaki, or progressive myoclonic epilepsy 1A. A VNTR can be present in the coding region or non-coding region. Moreover, a VNTR can be present in the 5′ untranslated region (UTR), promoter, intron, or 3′ UTR. The gene that includes, or is affected by, the VNTR can be, for example, PER3, MUC1, IL1RN, DUX4, DATI, MUC21, CEL, INS, DRD4, ACAN, ZFHX3, GPIBA, SERT, SERT, HIC1, MMP9, CSTB, or MAOA.

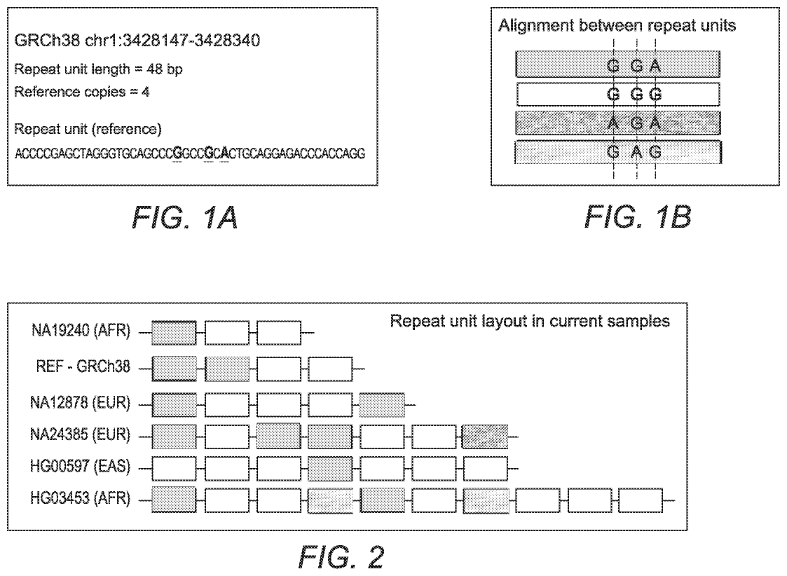

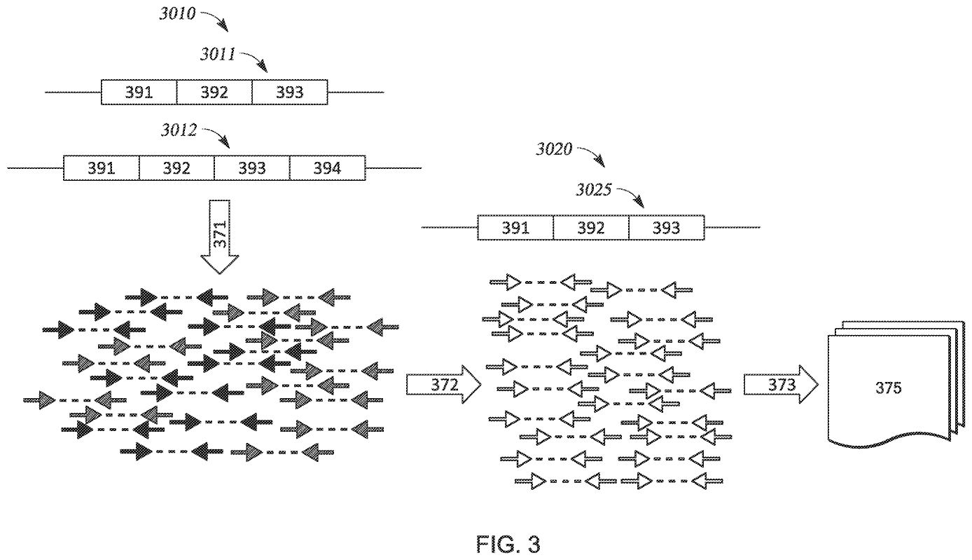

FIG. 1A, FIG. 1B and FIG. 2 show a non-limiting exemplary illustration of a VNTR in a reference sequence. FIG. 1A shows that a VNTR in the reference human genome GRCh38 is at chr1: 3428147-3428340. The repeat unit has a length of 48 bps. The reference sequence of the repeat unit ACCCCGAGCTAGGGTGCAGCCCGGCCGCACTGCAGGAGACCCACCAGG (SEQ ID NO: 1) in GRCh38. Different copies of the repeat unit in the VNTR (within a haplotype or across haplotypes) can vary, in particular at the three bases bolded and underlined. FIG. 1B shows that the three bases can be G, G, and A, respectively, in a first type or sequence of the repeat unit; G, G, and G, respectively, in a second type or sequence of the repeat unit; A, G, and A, respectively, in a third type or sequence of the repeat unit; and G, A, and G, respectively, in a fourth type or sequence of the repeat unit. FIG. 2 shows that the VNTR includes four copies of the repeat unit in GRCh38. The four copies include two copies of the first type followed by two copies of the second type. The five samples shown in FIG. 2 included three, five, seven, seven, and ten copies of the repeat unit, respectively. For sample NA19240 of a subject who is African, the VNTR included one copy of the first type followed by two copies of the second type. For sample NA12878 of a subject who is European, the VNTR included one copy of the first type, three copies of the second type, and one copy of the first type. For sample NA24385 of a subject who is European, the VNTR included one copy of the first type, one copy of the second type, two copies of the first type, two copies of the second type, and one copy of the third type. For sample HG00597 of a subject who is Eastern Asian, the VNTR included three copies of the second type, one copy of the first type, and three copies of the second type. For sample HG03453 of a subject who is African, the VNTR included one copy of the first type, two copies of the second type, one copy of the fourth type, one copy of the first type, one copy of the second type, one copy of the fourth type, and three copies of the second type. The examples discussed in connection with FIG. 1A, FIG. 1B and FIG. 2 pertain to homozygous variants where both alleles include the VNTR locus.

The difficulty of detecting VNTRs is multi-dimensional. The nature of the tandem repeats causes low mappability and high sequencing errors. Existing sequencing techniques (including, for example, using population haplotypes in the genome graph) suffer from low precision in detecting VNTRs due to the repetitive nature of VNTRs. Short-read sequencing technologies have a higher throughput compared to long-read sequencing technologies, but short sequencing reads often cannot cover the full length of most VNTRs. For example, around 29% of the VNTRs have additional repeats with total length greater than or equal to 150 bps in one individual. Due to the repetitive nature of VNTRs, correctly rebuilding VNTRs' haplotypes from short reads is difficult. With short sequencing reads, methods of detecting VNTRs may utilize the read sequences and some form of circular alignment (or wrap-around alignment) to infer the copy number changes in tandem repeats; however, these methods only allow for identification of small VNTRs (i.e., smaller than the read length). Abnormal fragment sizes—a read pair that maps beyond the normal distribution—have been used in the prior art to infer some classes of large structural variants such as large changes in VNTRs; however, some VNTRs may not be accurately detected by this approach if the VNTRs are shorter compared to the variance in the insert size of the sequencing reads. For example, VNTRs may not be accurately detected with paired-end sequencing reads, which have a high variance in insert size. Moreover, using existing methods, local reassembly of the VNTR sequences is difficult and often fails. Therefore, there is a need for improved methods for detecting and identifying VNTRs.

### Embodiments of Processes for Detecting Total Copy Numbers of Large Tandem Repeats

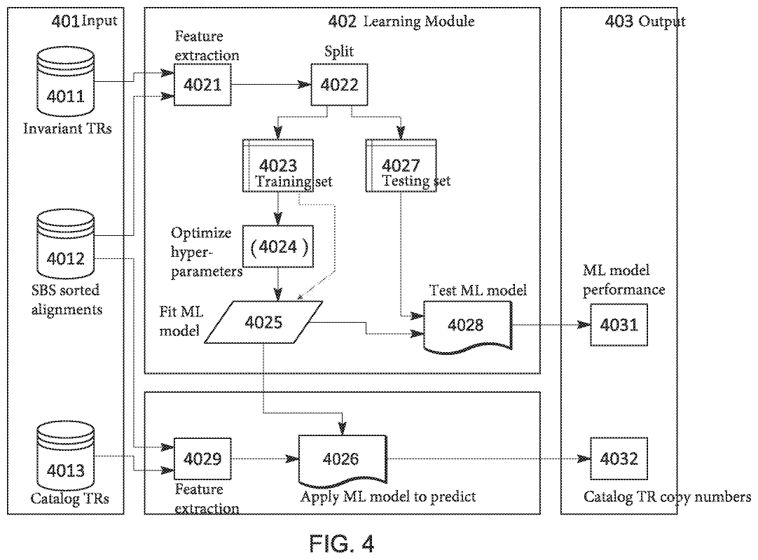

In some aspects, the disclosed technology relates to a method of predicting or estimating the total copy number of repeat units in a large tandem repeat (TR) in a sample using SBS technologies, such as short-read SBS technologies. One example is the short-read SBS sequencing technologies from Illumina, Inc. (San Diego, CA). FIG. 3 schematically illustrates an example process according to some embodiments of the disclosed technology utilizing read depth information from the SBS sequencing. As shown in FIG. 3, for the genome 3010 of a diploid organism, there may be two haplotypes with different copy numbers present in a particular TR region. For example, the maternal chromosome 3011 include 3 copies of repeat units (repeat unit 391, repeat unit 392 and repeat unit 393), and the paternal chromosome 3012 include 4 copies (repeat unit 391, repeat unit 392, repeat unit 393 and repeat unit 394). In such cases, the disclosed methods consider the total copy number of repeat units present in the particular TR region (i.e., the sum of the copy numbers of the two haplotypes).

In some embodiments, after subjecting the genome 3010 to pair-end sequencing 371 to generate SBS reads, and aligning (indicated by arrow 372 in FIG. 3) the SBS reads to the reference genome 3020 (including the reference TR locus 3025, which consists of repeat unit 391, repeat unit 392 and repeat unit 393), a machine learning (ML) model is used to learn the relationship between the total copy numbers of TRs and various features. In some embodiments, the disclosed technology builds the ML model on TR sequence features, such as pattern size and GC content, as well as sample-specific features or sequencing-read features, such as read depth, to predict the copy numbers of TRs in a given sample. In some embodiments, a set of predefined invariant TR regions are used to train the ML model, and the trained ML model is then used to predict/estimate (indicated by arrow 373 in FIG. 3) the copy numbers of a set of TR regions of interest with unknown copy numbers (the TR catalog) and generate a report 375.

To ensure accurate determination of the copy number of repeat units in a TR using the disclosed technology, in some embodiments, the array size of the TR may be at least one fragment size of the sequencer/the sequencing technology being used. Additionally, or alternatively, the minimum sequencing depth in the tandem repeat array may be 5×. Additionally or alternatively, the consensus pattern size (the size of a repeat unit in the reference TR locus in the reference genome) may be larger than about 5 bp, about 10 bp, about 15 bp or about 20 bp, such that changes in the TR array may be large enough to be detected.

In some embodiments, machine learning models use training data to learn the relationship between input and output data. The ML models may learn to generalize to new data that are similar to the training data and can thus make predictions about new data based on relationships learned from the training data. The relationships may approximate the underlying function between input and output data. The disclosed ML models can then be used to make predictions about trends or classify new data.

In some embodiments, the training (or fitting) of ML models involves an optimization process to iteratively refine and improve the ML model to minimize the degree of error between the predicted output and the true output. Each iteration step in the optimization process can improve the ML model's accuracy and lower the margin of error by adjusting the ML model's parameters. The iteration steps may be repeated until the optimal parameters that minimize the degree of error are found.

In some embodiments, disclosed methods build predictive ML models using supervised machine learning processes. The disclosed ML models may be regression models, such as linear regression, decision tree regressor, random forest regressor, or support vector regression, or classification models, such as logistic regression, k-nearest neighbors, decision tree classifier, support vector machine, random forest classifier, or XGBoost.

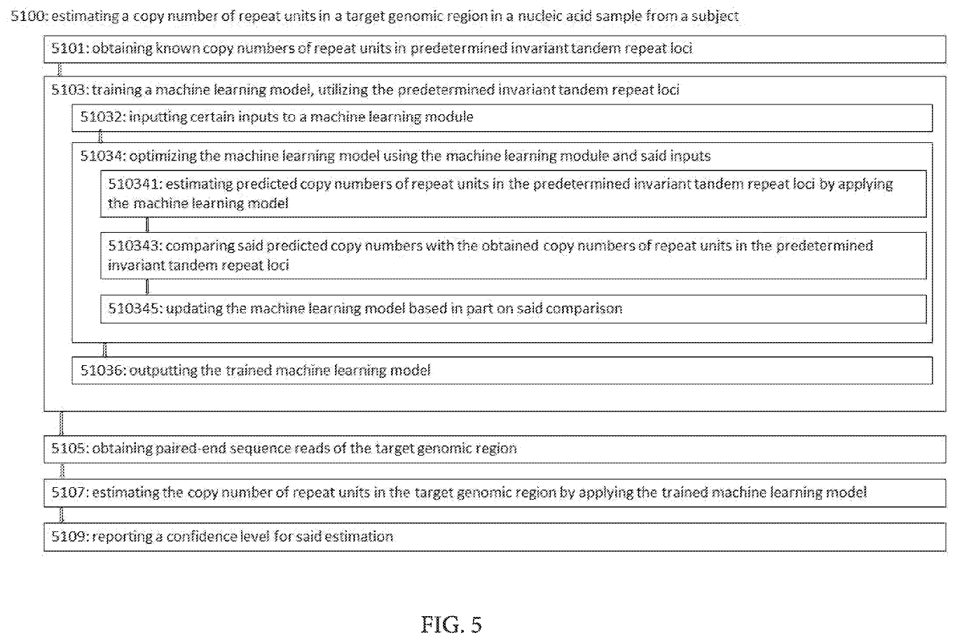

FIG. 4 is a block diagram that schematically illustrates an example machine learning module 402 for detecting and identifying TRs in a given sample of interest according to some embodiments of the disclosed technology. The machine learning module 402 may receive input data 401 and return/generate output data 403 based on the disclosed methods for detecting and determining total copy numbers of large tandem repeats, such as the example process described in connection with FIG. 3.

In some embodiments, input data 401 includes: invariant tandem repeats data 4011, which includes the list of tandem repeats that are approximately fixed or conserved in the human population, aligned paired-end sequencing reads data 4012 from sequencing of the given sample, where the data 4012 may be provided in BAM/CRAM format; and tandem repeat catalog 4013, which includes a list of tandem repeats of interest to be evaluated. In some embodiments, the invariant tandem repeats data 4011 can be obtained by searching over a large population to find a set of tandem repeats that almost always have the same genotype.

In some embodiments, the machine learning module 402 includes a feature extraction module 4021, which is used to extract features from the reference genome and the given sample in light of the invariant tandem repeats data 4011 and the aligned paired-end sequencing reads data 4012. In some embodiments, features extracted from the reference genome in light of the invariant tandem repeats data 4011 include: array size (the total size of a tandem repeat from start to end), consensus pattern size, and GC content of each of the invariant tandem repeats. In some embodiments, features extracted from the given sample in light of the aligned paired-end sequencing reads data 4012 include: the number of paired-end reads spanning the array (if any) of each of the invariant tandem repeats, the number of reads overlapping each of the invariant tandem repeats, the number of reads overlapping the start and end position (flanks) of each of the invariant tandem repeats, and the number of reads contained completely inside each of the invariant tandem repeat arrays.

In some embodiments, the machine learning module 402 further includes a feature extraction module 4029, which is used to extract features from the reference genome and the given sample in light of the tandem repeat catalog 4013 and the aligned paired-end sequencing reads data 4012. In some embodiments, features extracted from the reference genome in light of the tandem repeat catalog 4013 include: array size (the total size of a tandem repeat from start to end), consensus pattern size, and GC content of each of the TRs of interest. In some embodiments, features extracted from the given sample in light of the aligned paired-end sequencing reads data 4012 include: the number of paired-end reads spanning the array (if any) of each of the TRs of interest, the number of reads overlapping each of the TRs of interest, the number of reads overlapping the start and end position (flanks) of each of the TRs of interest, and the number of reads contained completely inside each of the TRs of interest.

To train a ML model specific for the input 401/the given sample, the machine learning module 402 may take the dataset processed by the feature extraction module 4021 and apply a module 4022 to split the dataset into a training set 4023 and a testing set 4027. For example, about 65%, 75% or 85% of the dataset may be used for training and the remaining portion may be used for testing. The machine learning module 402 may then utilize the training set 4023 to train the ML model (e.g., XGBoost) in module 4025, under a pre-determined best combination of hyper-parameters (i.e., parameters used to perform/control the machine learning process), and obtain a trained ML model that depicts a relationship between the known copy numbers of the invariant tandem repeats and the features extracted by module 4021. The machine learning module 402 may then utilize the testing set 4027 to test the trained ML model and measure/report its performance (e.g., RMSE) in module 4028. For example, the performance measured on the testing set 4027 can be used as a confidence level of the trained ML model. In some embodiments, the machine learning module 402 may output the performance as a portion 4031 of the output data 403.

To determine the best combination of hyper-parameters for the given sample of interest such that the learning process for the ML model is optimal (referred to as a hyper-parameter optimization or hyper-parameter tuning process), the machine learning module 402 may take one or more hyper-parameter tuning training sets 4023″ (which may be parts of the training set 4023) as input to module 4024 to find the best hyper-parameters for the ML model with cross-validation. The hyper-parameter optimization or hyper-parameter tuning process may utilize machine learning methods such as a search grid, evolution algorithms, random search, etc. In some embodiments, optimizing the ML model hyper-parameters include creating a hyper-parameter grid, finding the optimal hyper-parameters within the grid by using a cross-validation process on the one or more training sets 4023″, measuring the performance of the model (e.g., RMSE) on one or more validation sets 4027″ (which may be other parts of the training set 4023), and saving the best combination of hyper-parameters for future use (e.g., in module 4025). In some embodiments, determining the best combination of hyper-parameters for the given sample may occur “online”, utilizing the sequencing results of the given sample. To predict/estimate the copy numbers of the TRs of interest (in the tandem repeat catalog 4013) in the given sample, the machine learning module 402 may, in module 4026, obtain the trained ML model (which is specific for the input 401/the given sample) from module 4025, take the dataset processed by the feature extraction module 4029 and apply it to the trained ML model. In some embodiments, the machine learning module 402 may output the predicted/estimated copy numbers as a portion 4032 of the output data 403.

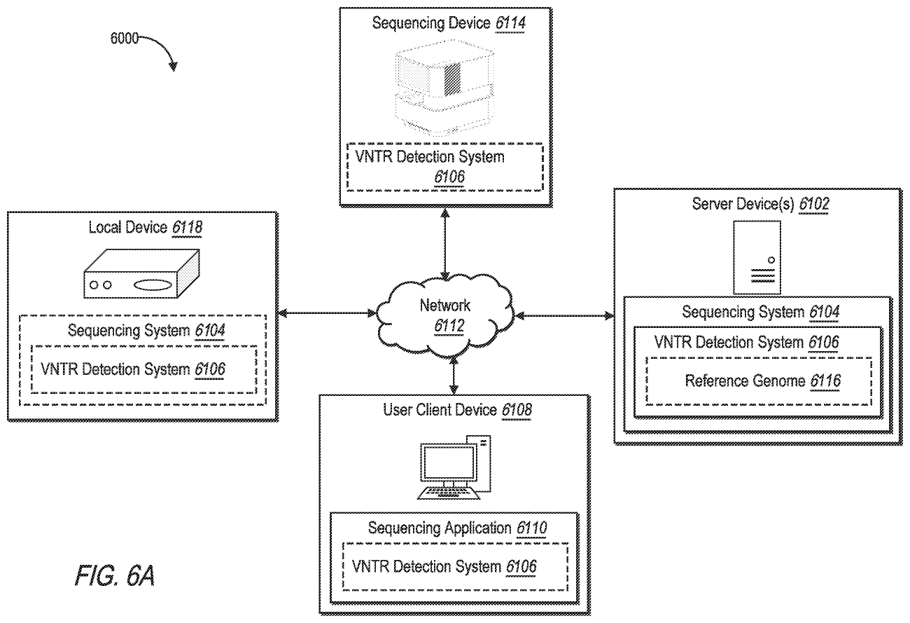

FIG. 5 is a flow chart that schematically illustrates an example method 5100 of estimating a copy number of repeat units in a target genomic region in a nucleic acid sample from a subject according to some embodiments of the disclosed technology, wherein the target genomic region corresponds to a tandem repeat locus. In some embodiments, the tandem repeat locus is a variable number tandem repeat (VNTR) locus. In some embodiments, a repeat unit in the tandem repeat locus is longer than about 700 base pairs in length. In some embodiments, the tandem repeat locus is part of a macrosatellite or a minisatellite. In some embodiments, the nucleic acid sample comprises both paternal and maternal nucleic acids for the subject. In some embodiments, the nucleic acid sample is extracted from cells, a cell-free DNA sample, an amniotic fluid, a blood sample, a biopsy sample, or any combination thereof, of the subject. In some embodiments, the subject is a human. In some embodiments, the example method 5100 may be performed by the machine learning module 402 described in connection with FIG. 4.

As shown in FIG. 5, the method 5100 of estimating a copy number of repeat units in a target genomic region may start from block 5101, wherein known copy numbers of repeat units in predetermined invariant tandem repeat loci are obtained. In some embodiments, the known copy numbers of repeat units in the predetermined invariant tandem repeat loci are obtained from a database or a reference genomic sequence. In some embodiments, each of the predetermined loci is invariant among subjects of the same species.

Next, the method 5100 may proceed to block 5103, wherein a machine learning model is trained to learn the relationship between: (a) the obtained copy numbers of repeat units in the predetermined invariant tandem repeat loci, and (b-i) specific features of the predetermined loci and (b-ii) specific features of training genomic regions in the nucleic acid sample from the subject that correspond to the predetermined loci. In some embodiments, the machine learning model is a XGBoost model, a regression model, a random forest model or a decision tree model. In some embodiments, (b-i), the specific features of the predetermined loci include: a size of the predetermined loci, a size of a repeat unit in the predetermined loci, or a GC content of the predetermined loci. In some embodiments, (b-ii), the specific features of the training genomic regions corresponding to the predetermined loci include: a number of paired-end sequence reads resulting from the training genomic regions that are spanning the predetermined loci, a number of paired-end sequence reads resulting from the training genomic regions that are overlapping the predetermined loci, a number of paired-end sequence reads resulting from the training genomic regions that are overlapping the start and end positions of the predetermined loci, or a number of paired-end sequence reads resulting from the training genomic regions that are contained inside the predetermined loci.

In some embodiments, training the machine learning model in block 5103 includes: step 51032 wherein the obtained copy numbers of repeat units in the predetermined invariant tandem repeat loci, the specific features of the predetermined loci, and the specific features of the training genomic regions corresponding to the predetermined loci are inputted to a machine learning module; step 51034 wherein the machine learning model is optimized using the machine learning module and said inputs; and step 51036 wherein the trained machine learning model is outputted, in which the model parameters characterizing the relationship between the obtained copy numbers and the specific features of the predetermined loci and the specific features of the training genomic regions are optimized.

In some embodiments, the step 51034 of optimizing the machine learning model may include: sub-step 510341 wherein predicted copy numbers of repeat units in the predetermined invariant tandem repeat loci is estimated by applying the machine learning model to the specific features of the predetermined loci and the specific features of the training genomic regions corresponding to the predetermined loci; sub-step 510343 wherein said predicted copy numbers are compared with the obtained copy numbers of repeat units in the predetermined invariant tandem repeat loci; and sub-step 510345 wherein the machine learning model is updated based in part on said comparison.

Next, the method 5100 may proceed to block 5105, wherein paired-end sequence reads of the target genomic region are obtained. In some embodiments, obtaining the paired-end sequence reads of the target genomic region includes: performing paired-end sequencing of the nucleic acid sample from the subject, aligning the paired-end sequence reads to a reference genomic sequence for the species of the subject, and obtaining the paired-end sequence reads that align to the tandem repeat locus on the reference genomic sequence. In some embodiments, each paired-end sequence read is about 100 base pairs to about 500 base pairs in length. In some embodiments, the paired-end sequence reads for the nucleic acid sample is generated by whole genome sequencing (WGS). In some embodiments, the paired-end sequence reads for the nucleic acid sample is generated by a next generation sequencing reaction. In some embodiments, each paired-end sequence read is obtained from a nucleic acid cluster on a solid substrate. In some embodiments, the nucleic acid cluster on the solid substrate is generated by a bridge amplification process.

Next, the method 5100 may proceed to block 5107, wherein the copy number of repeat units in the target genomic region is estimated, by applying the trained machine learning model to specific features of the tandem repeat locus and specific features of the target genomic region. In some embodiments, the specific features of the tandem repeat locus include: a size of the tandem repeat locus, a size of a repeat unit in the tandem repeat locus, or a GC content of the tandem repeat locus. In some embodiments, the specific features of the target genomic region include: a number of paired-end sequence reads resulting from the target genomic region that are spanning the tandem repeat locus, a number of paired-end sequence reads resulting from the target genomic region that are overlapping the tandem repeat locus, a number of paired-end sequence reads resulting from the target genomic region that are overlapping the start and end positions of the tandem repeat locus, or a number of paired-end sequence reads resulting from the target genomic region that are contained inside the tandem repeat locus.

Next, the method 5100 may proceed to block 5109, wherein a confidence level for said estimation is reported. In some embodiments, the confidence level is determined based in part on control genomic regions in the nucleic acid sample that correspond to control loci comprising invariant tandem repeats. In some embodiments, each of the control loci is invariant among subjects of the same species. In some embodiments, determining the confidence level includes: estimating predicted copy numbers of repeat units in the invariant tandem repeats of the control loci by applying the machine learning model to specific features of the control loci and specific features of the control genomic regions; comparing said predicted copy numbers with known copy numbers of repeat units in the invariant tandem repeats of the control loci; and determining the confidence level based on said comparison. In some embodiments, the known copy numbers of repeat units in the invariant tandem repeats of the control loci are obtained from a database or a reference genomic sequence. In some embodiments, the specific features of the control loci include: a size of the control loci, a size of a repeat unit in the control loci, or a GC content of the control loci. In some embodiments, the specific features of the control genomic regions include: a number of paired-end sequence reads resulting from the control genomic regions that are spanning the control loci, a number of paired-end sequence reads resulting from the control genomic regions that are overlapping the control loci, a number of paired-end sequence reads resulting from the control genomic regions that are overlapping the start and end positions of the control loci, or a number of paired-end sequence reads resulting from the control genomic regions that are contained inside the control loci.

### Examples

Table 1 shows an example subset of TR regions from the training set of invariant TRs. The features for each region are shown in the inner box, which include the pattern size, the GC content, the observed read count, the number of left flanking reads, the number of right flanking reads, and the number of spanning fragments. Through hyperparameter tuning and fitting the ML model, the trained ML model associates these features' values with the copy number of these regions, 3.

After the ML model is trained, it is applied on TR regions in the catalog with unknown copy numbers. In one example, the features for the region trf_803123, shown in Table 2, are similar to those for the invariant TR training subset shown in Table 1. As shown in Table 2, this region trf_803123 is predicted/estimated by the trained ML model to also have a copy number of 3.

TABLE 1

| Invariant | Pattern | GC | Observed | Left | Right | Spanning | Invariant |
|---|---|---|---|---|---|---|---|
| ID | size | content | read count | flank | flank | fragments | copy number |
| trf_614966 | 290 | 0.53 | 279 | 47 | 51 | 4 | 3 |
| trf_639008 | 293 | 0.56 | 282 | 43 | 36 | 0 | 3 |
| trf_671244 | 299 | 0.53 | 257 | 40 | 40 | 1 | 3 |
| trf_901208 | 293 | 0.55 | 288 | 40 | 37 | 1 | 3 |
| trf_903894 | 295 | 0.54 | 274 | 38 | 56 | 3 | 3 |
| trf_904877 | 286 | 0.54 | 266 | 45 | 39 | 5 | 3 |

TABLE 2

| | Pattern | GC | Observed | Left | Right | Spanning | Predicted |
|---|---|---|---|---|---|---|---|
| Catalog ID | size | content | read count | flank | flank | fragments | copy number |
| trf_803123 | 299 | 0.56 | 269 | 38 | 56 | 1 | 3 |

### Embodiments of Sequencing Systems

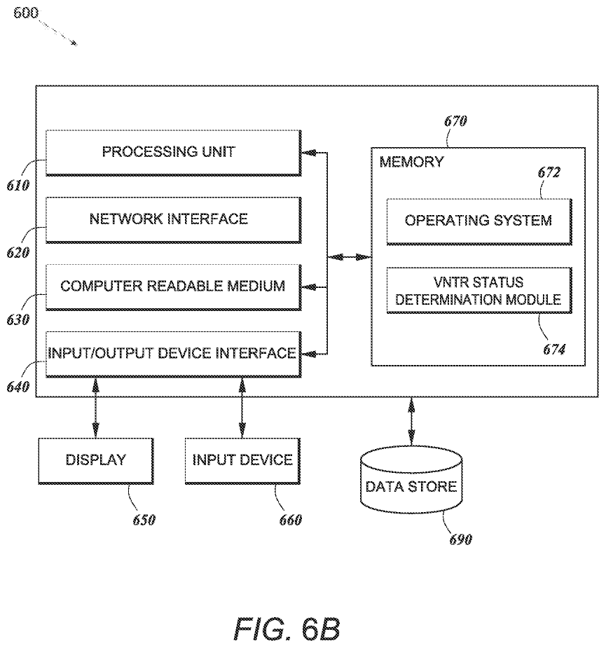

FIG. 6A is a block diagram of an exemplary sequencing system 6000 that may be used to perform or implement the disclosed technology, such as the example process described in connection with FIG. 3, the example machine learning module 402 described in connection with FIG. 4, or the example method 5100 described in connection with FIG. 5. For example, the sequencing system 6000 can be configured to determine a copy number of repeat units in a sample nucleic acid. The illustrative sequencing system 6000 may include a nucleic acid sequencer 6001, a non-transitory memory 6003 configured to store executable instructions, and a hardware processor 6005 in communication with the nucleic acid sequencer 6001 and the non-transitory memory 6003. The hardware processor 6005 may be programmed by the executable instructions to perform the methods disclosed herein.

In some embodiments, the non-transitory memory 6003 is configured to store the reference sequence. In some embodiments, the hardware processor 6005 is configured to obtain the reference sequence from an external database. In some embodiments, the hardware processor 6005 is configured to receive paired-end sequence reads from the nucleic acid sequencer 6001. In some embodiments, the hardware processor 6005 is configured to control the nucleic acid sequencer 6001 to perform sequencing of the sample nucleic acid. In some embodiments, the hardware processor 6005 is configured to control the nucleic acid sequencer 6001 to perform additional sequencing of the sample nucleic acid based on the determined most likely copy number of repeat units in the sample nucleic acid 6001. In some embodiments, the hardware processor 6005 is configured to output, on a display, the most likely copy number of repeat units in the sample nucleic acid.

FIG. 6B is a block diagram of an exemplary computing device 600 that may be used in connection with the illustrative sequencing system 6000 of FIG. 6A. The computing device Error! Reference source not found.00 may be configured to determine a VNTR status, such as identifying a VNTR. The general architecture of the computing device Error! Reference source not found.00 depicted in FIG. 6B includes an arrangement of computer hardware and software components. The computing device Error! Reference source not found.00 may include many more (or fewer) elements than those shown in FIG. 6B. It is not necessary, however, that all of these generally conventional elements be shown in order to provide an enabling disclosure. As illustrated, the computing device Error! Reference source not found.00 includes a processing unit Error! Reference source not found. 10, a network interface Error! Reference source not found.20, a computer readable medium drive Error! Reference source not found.30, an input/output device interface Error! Reference source not found.40, a display Error! Reference source not found.50, and an input device Error! Reference source not found.60, all of which may communicate with one another by way of a communication bus. The network interface Error! Reference source not found.20 may provide connectivity to one or more networks or computing systems. The processing unit Error! Reference source not found.10 may thus receive information and instructions from other computing systems or services via a network. The processing unit Error! Reference source not found.10 may also communicate to and from memory Error! Reference source not found. 70 and further provide output information for an optional display Error! Reference source not found.50 via the input/output device interface Error! Reference source not found.40. The input/output device interface Error! Reference source not found.40 may also accept input from the optional input device Error! Reference source not found.60, such as a keyboard, mouse, digital pen, microphone, touch screen, gesture recognition system, voice recognition system, gamepad, accelerometer, gyroscope, or other input device.

The memory Error! Reference source not found.70 may contain computer program instructions (grouped as modules or components in some embodiments) that the processing unit Error! Reference source not found.10 executes in order to implement one or more embodiments. The memory Error! Reference source not found.70 generally includes RAM, ROM and/or other persistent, auxiliary or non-transitory computer-readable media. The memory Error! Reference source not found.70 may store an operating system Error! Reference source not found.72 that provides computer program instructions for use by the processing unit Error! Reference source not found.10 in the general administration and operation of the computing device Error! Reference source not found.00. The memory Error! Reference source not found.70 may further include computer program instructions and other information for implementing aspects of the present disclosure.

For example, in one embodiment, the memory Error! Reference source not found.70 includes a VNTR status determination module Error! Reference source not found.74 for determining a VNTR status. The VNTR status determination module Error! Reference source not found.74 can perform the methods disclosed herein. In addition, memory Error! Reference source not found.70 may include or communicate with the data store Error! Reference source not found.90 and/or one or more other data stores that store one or more inputs, one or more outputs, and/or one or more results (including intermediate results) of determining a VNTR status of the present disclosure, such the long reads, the short reads, and the VNTR status determined.

In some embodiments, the disclosed systems and methods may involve approaches for shifting or distributing certain sequence data analysis features and sequence data storage to a cloud computing environment or cloud-based network. User interaction with sequencing data, genome data, or other types of biological data may be mediated via a central hub that stores and controls access to various interactions with the data. In some embodiments, the cloud computing environment may also provide sharing of protocols, analysis methods, libraries, sequence data as well as distributed processing for sequencing, analysis, and reporting. In some embodiments, the cloud computing environment facilitates modification or annotation of sequence data by users. In some embodiments, the systems and methods may be implemented in a computer browser, on-demand or on-line.

In some embodiments, software written to perform the methods as described herein is stored in some form of computer readable medium, such as memory, CD-ROM, DVD-ROM, memory stick, flash drive, hard drive, SSD hard drive, server, mainframe storage system and the like.

In some embodiments, the methods may be written in any of various suitable programming languages, for example compiled languages such as C, C#, C++, Fortran, and Java. Other programming languages could be script languages, such as Perl, MatLab, SAS, SPSS, Python, Ruby, Pascal, Delphi, R and PHP. In some embodiments, the methods are written in C, C#, C++, Fortran, Java, Perl, R, Java or Python. In some embodiments, the method may be an independent application with data input and data display modules. Alternatively, the method may be a computer software product and may include classes wherein distributed objects comprise applications including computational methods as described herein.

In some embodiments, the methods may be incorporated into pre-existing data analysis software, such as that found on sequencing instruments. Software comprising computer implemented methods as described herein are installed either onto a computer system directly, or are indirectly held on a computer readable medium and loaded as needed onto a computer system. Further, the methods may be located on computers that are remote to where the data is being produced, such as software found on servers and the like that are maintained in another location relative to where the data is being produced, such as that provided by a third party service provider.

An assay instrument, desktop computer, laptop computer, or server which may contain a processor in operational communication with accessible memory comprising instructions for implementation of systems and methods. In some embodiments, a desktop computer or a laptop computer is in operational communication with one or more computer readable storage media or devices and/or outputting devices. An assay instrument, desktop computer and a laptop computer may operate under a number of different computer based operational languages, such as those utilized by Apple based computer systems or PC based computer systems. An assay instrument, desktop and/or laptop computers and/or server system may further provide a computer interface for creating or modifying experimental definitions and/or conditions, viewing data results and monitoring experimental progress. In some embodiments, an outputting device may be a graphic user interface such as a computer monitor or a computer screen, a printer, a hand-held device such as a personal digital assistant (i.e., PDA, Blackberry, iPhone), a tablet computer (e.g., iPAD), a hard drive, a server, a memory stick, a flash drive and the like.

A computer readable storage device or medium may be any device such as a server, a mainframe, a supercomputer, a magnetic tape system and the like. In some embodiments, a storage device may be located onsite in a location proximate to the assay instrument, for example adjacent to or in close proximity to, an assay instrument. For example, a storage device may be located in the same room, in the same building, in an adjacent building, on the same floor in a building, on different floors in a building, etc. in relation to the assay instrument. In some embodiments, a storage device may be located off-site, or distal, to the assay instrument. For example, a storage device may be located in a different part of a city, in a different city, in a different state, in a different country, etc. relative to the assay instrument. In embodiments where a storage device is located distal to the assay instrument, communication between the assay instrument and one or more of a desktop, laptop, or server is typically via Internet connection, either wireless or by a network cable through an access point. In some embodiments, a storage device may be maintained and managed by the individual or entity directly associated with an assay instrument, whereas in other embodiments a storage device may be maintained and managed by a third party, typically at a distal location to the individual or entity associated with an assay instrument. In embodiments as described herein, an outputting device may be any device for visualizing data.

An assay instrument, desktop, laptop and/or server system may be used itself to store and/or retrieve computer implemented software programs incorporating computer code for performing and implementing computational methods as described herein, data for use in the implementation of the computational methods, and the like. One or more of an assay instrument, desktop, laptop and/or server may comprise one or more computer readable storage media for storing and/or retrieving software programs incorporating computer code for performing and implementing computational methods as described herein, data for use in the implementation of the computational methods, and the like. Computer readable storage media may include, but is not limited to, one or more of a hard drive, a SSD hard drive, a CD-ROM drive, a DVD-ROM drive, a floppy disk, a tape, a flash memory stick or card, and the like. Further, a network including the Internet may be the computer readable storage media. In some embodiments, computer readable storage media refers to computational resource storage accessible by a computer network via the Internet or a company network offered by a service provider rather than, for example, from a local desktop or laptop computer at a distal location to the assay instrument.

In some embodiments, computer readable storage media for storing and/or retrieving computer implemented software programs incorporating computer code for performing and implementing computational methods as described herein, data for use in the implementation of the computational methods, and the like, is operated and maintained by a service provider in operational communication with an assay instrument, desktop, laptop and/or server system via an Internet connection or network connection.

In some embodiments, a hardware platform for providing a computational environment comprises a processor (i.e., CPU) wherein processor time and memory layout such as random access memory (i.e., RAM) are systems considerations. For example, smaller computer systems offer inexpensive, fast processors and large memory and storage capabilities. In some embodiments, graphics processing units (GPUs) can be used. In some embodiments, hardware platforms for performing computational methods as described herein comprise one or more computer systems with one or more processors. In some embodiments, smaller computer are clustered together to yield a supercomputer network.

In some embodiments, computational methods as described herein are carried out on a collection of inter- or intra-connected computer systems (i.e., grid technology) which may run a variety of operating systems in a coordinated manner. For example, the CONDOR framework (University of Wisconsin-Madison) and systems available through United Devices are exemplary of the coordination of multiple stand-alone computer systems for the purpose dealing with large amounts of data. These systems may offer Perl interfaces to submit, monitor and manage large sequence analysis jobs on a cluster in serial or parallel configurations.

### Samples

In some embodiments, the sample comprises or consists of a purified or isolated polynucleotide derived from a tissue sample, a biological fluid sample, a cell sample, and the like. Suitable biological fluid samples include, but are not limited to blood, plasma, serum, sweat, tears, sputum, urine, sputum, ear flow, lymph, saliva, cerebrospinal fluid, ravages, bone marrow suspension, vaginal flow, trans-cervical lavage, brain fluid, ascites, milk, secretions of the respiratory, intestinal and genitourinary tracts, amniotic fluid, milk, and leukophoresis samples. In some embodiments, the sample is a sample that is easily obtainable by non-invasive procedures, e.g., blood, plasma, serum, sweat, tears, sputum, urine, sputum, ear flow, saliva or feces. In certain embodiments the sample is a peripheral blood sample, or the plasma and/or serum fractions of a peripheral blood sample. In other embodiments, the biological sample is a swab or smear, a biopsy specimen, or a cell culture. In another embodiment, the sample is a mixture of two or more biological samples, e.g., a biological sample can comprise two or more of a biological fluid sample, a tissue sample, and a cell culture sample. As used herein, the terms “blood,” “plasma” and “serum” expressly encompass fractions or processed portions thereof. Similarly, where a sample is taken from a biopsy, swab, smear, etc., the “sample” expressly encompasses a processed fraction or portion derived from the biopsy, swab, smear, etc.

In certain embodiments, samples can be obtained from sources, including, but not limited to, samples from different individuals, samples from different developmental stages of the same or different individuals, samples from different diseased individuals (e.g., individuals with cancer or suspected of having a genetic disorder), normal individuals, samples obtained at different stages of a disease in an individual, samples obtained from an individual subjected to different treatments for a disease, samples from individuals subjected to different environmental factors, samples from individuals with predisposition to a pathology, samples individuals with exposure to an infectious disease agent, and the like.

In one illustrative, but non-limiting embodiment, the sample is a maternal sample that is obtained from a pregnant female, for example a pregnant woman. The maternal sample can be a tissue sample, a biological fluid sample, or a cell sample. In another illustrative, but non-limiting embodiment, the maternal sample is a mixture of two or more biological samples, e.g., the biological sample can comprise two or more of a biological fluid sample, a tissue sample, and a cell culture sample.

In certain embodiments samples can also be obtained from in vitro cultured tissues, cells, or other polynucleotide-containing sources. The cultured samples can be taken from sources including, but not limited to, cultures (e.g., tissue or cells) maintained in different media and conditions (e.g., pH, pressure, or temperature), cultures (e.g., tissue or cells) maintained for different periods of length, cultures (e.g., tissue or cells) treated with different factors or reagents (e.g., a drug candidate, or a modulator), or cultures of different types of tissue and/or cells.

In some embodiments, the use of the disclosed sequencing technology does not involve the preparation of sequencing libraries. In other embodiments, the sequencing technology contemplated herein involve the preparation of sequencing libraries. In one illustrative approach, sequencing library preparation involves the production of a random collection of adapter-modified DNA fragments (e.g., polynucleotides) that are ready to be sequenced.

Sequencing libraries of polynucleotides can be prepared from DNA or RNA, including equivalents, analogs of either DNA or cDNA, for example, DNA or cDNA that is complementary or copy DNA produced from an RNA template, by the action of reverse transcriptase. The polynucleotides may originate in double-stranded form (e.g., dsDNA such as genomic DNA fragments, cDNA, PCR amplification products, and the like) or, in certain embodiments, the polynucleotides may originate in single-stranded form (e.g., ssDNA, RNA, etc.) and have been converted to dsDNA form. By way of illustration, in certain embodiments, single stranded mRNA molecules may be copied into double-stranded cDNAs suitable for use in preparing a sequencing library. The precise sequence of the primary polynucleotide molecules is generally not material to the method of library preparation, and may be known or unknown. In one embodiment, the polynucleotide molecules are DNA molecules. More particularly, in certain embodiments, the polynucleotide molecules represent the entire genetic complement of an organism or substantially the entire genetic complement of an organism, and are genomic DNA molecules (e.g., cellular DNA, cell free DNA (cfDNA), etc.), that typically include both intron sequence and exon sequence (coding sequence), as well as non-coding regulatory sequences such as promoter and enhancer sequences. In certain embodiments, the primary polynucleotide molecules comprise human genomic DNA molecules, e.g., cfDNA molecules present in peripheral blood of a pregnant subject.

Methods of isolating nucleic acids from biological sources may differ depending upon the nature of the source. One of skill in the art can readily isolate nucleic acids from a source as needed for the method described herein. In some instances, it can be advantageous to fragment large nucleic acid molecules (e.g. cellular genomic DNA) in the nucleic acid sample to obtain polynucleotides in the desired size range. Fragmentation can be random, or it can be specific, as achieved, for example, using restriction endonuclease digestion. Methods for random fragmentation may include, for example, limited DNase digestion, alkali treatment and physical shearing. Fragmentation can also be achieved by any of a number of methods known to those of skill in the art. For example, fragmentation can be achieved by mechanical means including, but not limited to nebulization, sonication and hydroshear.

In some embodiments, sample nucleic acids are obtained from as cfDNA, which is not subjected to fragmentation. For example, cfDNA, typically exists as fragments of less than about 300 base pairs and consequently, fragmentation is not typically necessary for generating a sequencing library using cfDNA samples.

Typically, whether polynucleotides are forcibly fragmented (e.g., fragmented in vitro), or naturally exist as fragments, they are converted to blunt-ended DNA having 5′-phosphates and 3′-hydroxyl. Protocols for sequencing may instruct users to end-repair sample DNA, to purify the end-repaired products prior to dA-tailing, and to purify the dA-tailing products prior to the adaptor-ligating steps of the library preparation.

In various embodiments, verification of the integrity of the samples and sample tracking can be accomplished by sequencing mixtures of sample genomic nucleic acids, e.g., cfDNA, and accompanying marker nucleic acids that have been introduced into the samples, e.g., prior to processing.

### Definitions

Unless defined otherwise, technical and scientific terms used herein have the same meaning as commonly understood by one of ordinary skill in the art to which the present disclosure belongs. See, e.g. Singleton et al., Dictionary of Microbiology and Molecular Biology 2nd ed., J. Wiley & Sons (New York, NY 1994); Sambrook et al., Molecular Cloning, A Laboratory Manual, Cold Spring Harbor Press (Cold Spring Harbor, NY 1989). For purposes of the present disclosure, the following terms are defined below.

As used herein, a “nucleotide” includes a nitrogen containing heterocyclic base, a sugar, and one or more phosphate groups. Nucleotides are monomeric units of a nucleic acid sequence. Examples of nucleotides include, for example, ribonucleotides or deoxyribonucleotides. In ribonucleotides (RNA), the sugar is a ribose, and in deoxyribonucleotides (DNA), the sugar is a deoxyribose, i.e., a sugar lacking a hydroxyl group that is present at the 2′ position in ribose. The nitrogen containing heterocyclic base can be a purine base or a pyrimidine base. Purine bases include adenine (A) and guanine (G), and modified derivatives or analogs thereof. Pyrimidine bases include cytosine (C), thymine (T), and uracil (U), and modified derivatives or analogs thereof. The C-1 atom of deoxyribose is bonded to N-1 of a pyrimidine or N-9 of a purine. The phosphate groups may be in the mono-, di-, or tri-phosphate form. These nucleotides may be natural nucleotides, but it is to be further understood that non-natural nucleotides, modified nucleotides or analogs of the aforementioned nucleotides can also be used.

As used herein, “nucleobase” is a heterocyclic base such as adenine, guanine, cytosine, thymine, uracil, inosine, xanthine, hypoxanthine, or a heterocyclic derivative, analog, or tautomer thereof. A nucleobase can be naturally occurring or synthetic. Non-limiting examples of nucleobases are adenine, guanine, thymine, cytosine, uracil, xanthine, hypoxanthine, 8-azapurine, purines substituted at the 8 position with methyl or bromine, 9-oxo-N6-methyladenine, 2-aminoadenine, 7-deazaxanthine, 7-deazaguanine, 7-deaza-adenine, N4-ethanocytosine, 2,6-diaminopurine, N6-ethano-2,6-diaminopurine, 5-methylcytosine, 5-(C3-C6)-alkynylcytosine, 5-fluorouracil, 5-bromouracil, thiouracil, pseudoisocytosine, 2-hydroxy-5-methyl-4-triazolopyridine, isocytosine, isoguanine, inosine, 7,8-dimethylalloxazine, 6-dihydrothymine, 5,6-dihydrouracil, 4-methyl-indole, ethenoadenine and the non-naturally occurring nucleobases described in U.S. Pat. Nos. 5,432,272 and 6,150,510 and PCT applications WO 92/002258, WO 93/10820, WO 94/22892, and WO 94/24144, and Fasman (“Practical Handbook of Biochemistry and Molecular Biology”, pp. 385-394, 1989, CRC Press, Boca Raton, LO), all herein incorporated by reference in their entireties.

The term “nucleic acid” or “polynucleotide” refers to a deoxyribonucleotide or ribonucleotide polymer in either single- or double-stranded form, and unless otherwise limited, encompasses known analogs of natural nucleotides that hybridize to nucleic acids in manner similar to naturally occurring nucleotides, such as peptide nucleic acids (PNAs) and phosphorothioate DNA. Unless otherwise indicated, a particular nucleic acid sequence includes the complementary sequence thereof. Nucleotides include, but are not limited to, ATP, dATP, CTP, dCTP, GTP, dGTP, UTP, TTP, dUTP, 5-methyl-CTP, 5-methyl-dCTP, ITP, dITP, 2-amino-adenosine-TP, 2-amino-deoxyadenosine-TP, 2-thiothymidine triphosphate, pyrrolo-pyrimidine triphosphate, and 2-thiocytidine, as well as the alphathiotriphosphates for all of the above, and 2′-O-methyl-ribonucleotide triphosphates for all the above bases. Modified bases include, but are not limited to, 5-Br-UTP, 5-Br-dUTP, 5-F-UTP, 5-F-dUTP, S-propynyl dCTP, and 5-propynyl-dUTP.

The term “primer,” as used herein refers to an isolated oligonucleotide that is capable of acting as a point of initiation of synthesis when placed under conditions inductive to synthesis of an extension product (e.g., the conditions include nucleotides, an inducing agent such as DNA polymerase, and a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer, use of the method, and the parameters used for primer design.

As used herein the term “chromosome” refers to the heredity-bearing gene carrier of a living cell, which is derived from chromatin strands comprising DNA and protein components (especially histones). The conventional internationally recognized individual human genome chromosome numbering system is employed herein.

A “genome” refers to the complete genetic information of an organism or virus, expressed in nucleic acid sequences.

As used herein, the term “reference genome” or “reference sequence” refers to any particular known genome sequence, whether partial or complete, of any organism or virus which may be used to reference identified sequences from a subject. For example, a reference genome used for human subjects as well as many other organisms is found at the National Center for Biotechnology Information at ncbi.nlm.nih.gov. In various embodiments, the reference sequence is significantly larger than the reads that are aligned to it. For example, it may be at least about 100 times larger, or at least about 1000 times larger, or at least about 10,000 times larger, or at least about 105 times larger, or at least about 106 times larger, or at least about 107 times larger. In one example, the reference sequence is that of a full-length genome. Such sequences may be referred to as genomic reference sequences. For example, the reference sequence can be a reference human genome sequence, such as hg19 or hg38. In another example, the reference sequence is limited to a specific human chromosome such as chromosome 13. In some embodiments, a reference Y chromosome is the Y chromosome sequence from human genome version hg19. Such sequences may be referred to as chromosome reference sequences. Other examples of reference sequences include genomes of other species, as well as chromosomes, sub-chromosomal regions (such as strands), etc., of any species. In various embodiments, the reference sequence is a consensus sequence or other combination derived from multiple individuals. However, in certain applications, the reference sequence may be taken from a particular individual.

The term “nucleic acid sample” herein refers to a sample, typically derived from a biological fluid, cell, tissue, organ, or organism, comprising a nucleic acid or a mixture of nucleic acids comprising at least one nucleic acid sequence that is to be screened for copy number variation. In certain embodiments the nucleic acid sample comprises at least one nucleic acid sequence whose copy number is suspected of having undergone variation. Such samples may include, but are not limited to sputum/oral fluid, amniotic fluid, blood, a blood fraction, or fine needle biopsy samples (e.g., surgical biopsy, fine needle biopsy, etc.), urine, peritoneal fluid, pleural fluid, and the like. Although the sample is often taken from a human subject (e.g., patient), the sample may be from any mammal, including, but not limited to dogs, cats, horses, goats, sheep, cattle, pigs, etc. The sample may be used directly as obtained from the biological source or following a pretreatment to modify the character of the sample. For example, such pretreatment may include preparing plasma from blood, diluting viscous fluids and so forth. Methods of pretreatment may also involve, but are not limited to, filtration, precipitation, dilution, distillation, mixing, centrifugation, freezing, lyophilization, concentration, amplification, nucleic acid fragmentation, inactivation of interfering components, the addition of reagents, lysing, etc. If such methods of pretreatment are employed with respect to the sample, such pretreatment methods are typically such that the nucleic acid(s) of interest remain in the test sample, sometimes at a concentration proportional to that in an untreated test sample (e.g., namely, a sample that is not subjected to any such pretreatment method(s)). Such “treated” or “processed” samples are still considered to be biological “test” samples with respect to the methods described herein.

The term “subject” herein refers to a human subject as well as a non-human subject such as a mammal, an invertebrate, a vertebrate, a fungus, a yeast, a bacterium, and a virus. Although the examples herein concern humans and the language is primarily directed to human concerns, the concepts disclosed herein are applicable to genomes from any plant or animal, and are useful in the fields of veterinary medicine, animal sciences, research laboratories and such.

The term “condition” or “medical condition” is used herein as a broad term that includes all diseases and disorders, but can include injuries and normal health situations, such as pregnancy, that might affect a person's health, benefit from medical assistance, or have implications for medical treatments.

As used herein, the term “cluster” or “clump” refers to a group of molecules, e.g., a group of DNA, or a group of signals. In some embodiments, the signals of a cluster are derived from different features. In some embodiments, a signal clump represents a physical region covered by one amplified oligonucleotide. Each signal clump could be ideally observed as several signals. Accordingly, duplicate signals could be detected from the same clump of signals. In some embodiments, a cluster or clump of signals can comprise one or more signals or spots that correspond to a particular feature. When used in connection with microarray devices or other molecular analytical devices, a cluster can comprise one or more signals that together occupy the physical region occupied by an amplified oligonucleotide (or other polynucleotide or polypeptide with a same or similar sequence). For example, where a feature is an amplified oligonucleotide, a cluster can be the physical region covered by one amplified oligonucleotide. In other embodiments, a cluster or clump of signals need not strictly correspond to a feature. For example, spurious noise signals may be included in a signal cluster but not necessarily be within the feature area. For example, a cluster of signals from four cycles of a sequencing reaction could comprise at least four signals.

The term “next generation sequencing (NGS)” herein refers to sequencing methods that allow for massively parallel sequencing of clonally amplified molecules and of single nucleic acid molecules. Non-limiting examples of NGS include sequencing-by-synthesis using reversible dye terminators, and sequencing-by-ligation.

The term “read” or “sequence read” (or sequencing reads) refer to a sequence obtained from a portion of a nucleic acid sample. A read may be represented by a string of nucleotides sequenced from any part or all of a nucleic acid molecule. Typically, though not necessarily, a read represents a short sequence of contiguous base pairs in the sample. The read may be represented symbolically by the base pair sequence (in A, T, C, or G) of the sample portion. It may be stored in a memory device and processed as appropriate to determine whether it matches a reference sequence or meets other criteria. A read may be obtained directly from a sequencing apparatus or indirectly from stored sequence information concerning the sample. In some cases, a read is a DNA sequence of sufficient length (e.g., at least about 25 bp) that can be used to identify a larger sequence or region, e.g., that can be aligned and specifically assigned to a chromosome or genomic region or gene. For example, a sequence read may be a short string of nucleotides (e.g., 20-150 bases) sequenced from a nucleic acid fragment, a short string of nucleotides at one or both ends of a nucleic acid fragment, or the sequencing of the entire nucleic acid fragment that exists in the biological sample. A sequence read may be obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification. Sequence reads can be generated by techniques such as sequencing by synthesis, sequencing by binding, or sequencing by ligation. Sequence reads can be generated using instruments such as MINISEQ, MISEQ, NEXTSEQ, HISEQ, and NOVASEQ sequencing instruments from Illumina, Inc. (San Diego, CA).

The term “sequencing depth,” as used herein, generally refers to the number of times a locus is covered by a sequence read aligned to the locus. The locus may be as small as a nucleotide, or as large as a chromosome arm, or as large as the entire genome. Sequencing depth can be expressed as 50×, 100×, etc., where “×” refers to the number of times a locus is covered with a sequence read. Sequencing depth can also be applied to multiple loci, or the whole genome, in which case× can refer to the mean number of times the loci or the haploid genome, or the whole genome, respectively, is sequenced. When a mean depth is quoted, the actual depth for different loci included in the dataset spans over a range of values. Ultra-deep sequencing can refer to at least 100× in sequencing depth.

The term “coverage” refers to the abundance of sequence tags mapped to a defined sequence. Coverage can be quantitatively indicated by sequence tag density (or count of sequence tags), sequence tag density ratio, normalized coverage amount, adjusted coverage values, etc. In some cases, “effective read coverage” of a chromosome is defined as the actual amount of bases covered by reads. Sequencing depth, which refers to the expected coverage of nucleotides by reads, is computed based on the assumption that reads are synthesized uniformly across chromosomes. In reality, read coverage across genomes is not uniform. Although a coverage of 10×, for example, means a nucleotide is covered 10 times on average, in certain parts of a genome, nucleotides are covered much more or much less. One factor that influences coverage is the ability of a read aligner to align reads to genomes. If a part of a genome is complex, e.g. having many repeats, aligners might have troubles aligning reads to that region, resulting in low coverage.

As used herein, the terms “aligned,” “alignment,” or “aligning” refer to the process of comparing a read or tag to a reference sequence and thereby determining the likelihood of the reference sequence contains the read sequence. If the reference sequence contains the read, the read may be mapped to the reference sequence or, in certain embodiments, to a particular location in the reference sequence. For example, the alignment of a read to the reference sequence for human chromosome 13 will tell the likelihood of the read is present in the reference sequence for chromosome 13. In some cases, an alignment additionally indicates a location where the read or tag maps to in the reference sequence. For example, if the reference sequence is the whole human genome sequence, an alignment may indicate that a read is present on chromosome 13, and may further indicate that the read is on a particular strand and/or site of chromosome 13. A “site” may be a unique position on a polynucleotide sequence or a reference genome (i.e. chromosome ID, chromosome position and orientation). In some embodiments, a site may provide a position for a residue, a sequence tag, or a segment on a sequence.

Aligned reads or tags are one or more sequences that are identified as a match in terms of the order of their nucleic acid molecules to a known sequence from a reference genome. Alignment can be done manually, although it is typically implemented by a computer algorithm, as it would be impossible to align reads in a reasonable time period for implementing the methods disclosed herein. The matching of a sequence read in aligning can be a 100% sequence match or less than 100% (non-perfect match).

Alignment may be performed by modifications and/or combinations of methods such as Burrows-Wheeler Aligner (BWA), ISAAC, BarraCUDA, BFAST, BLASTN, BLAT, Bowtie, CASHX, Cloudburst, CUDA-EC, CUSHAW, CUSHAW2, CUSHAW2-GPU, drFAST, ELAND, ERNE, GNUMAP, GEM, GensearchNGS, GMAP and GSNAP, Geneious Assembler, LAST, MAQ, mrFAST and mrsFAST, MOM, MOSAIK, MPscan, Novoaligh & NovoalignCS, NextGENe, Omixon, PALMapper, Partek, PASS, PerM, PRIMEX, QPalma, RazerS, REAL, cREAL, RMAP, rNA, RT Investigator, Segemehl, SeqMap, Shrec, SHRIMP, SLIDER, SOAP, SOAP2, SOAP3 and SOAP3-dp, SOCS, SSAHA and SSAHA2, Stampy, STORM, Subread and Subjunc, Taipan, UGENE, VelociMapper, XpressAlign, and ZOOM.

The term “mapping” used herein refers to specifically assigning a sequence read to a larger sequence, e.g., a reference genome, by alignment.

A “genetic variation” or “genetic alteration” refers to a particular genotype present in certain individuals, and often a genetic variation is present in a statistically significant sub-population of individuals. The presence or absence of a genetic variance can be determined using a method or apparatus described herein. In certain embodiments, the presence or absence of one or more genetic variations is determined according to an outcome provided by methods and apparatuses described herein. In some embodiments, a genetic variation is a chromosome abnormality (e.g., aneuploidy), partial chromosome abnormality or mosaicism, each of which is described in greater detail herein. Non-limiting examples of genetic variations include one or more deletions (e.g., micro-deletions), duplications (e.g., micro-duplications), insertions, mutations, polymorphisms (e.g., single-nucleotide polymorphisms), fusions, repeats (e.g., short tandem repeats), distinct methylation sites, distinct methylation patterns, the like and combinations thereof. An insertion, repeat, deletion, duplication, mutation or polymorphism can be of any length, and in some embodiments, is about 1 base or base pair (bp) to about 250 megabases (Mb) in length. In some embodiments, an insertion, repeat, deletion, duplication, mutation or polymorphism is about 1 base or base pair (bp) to about 1,000 kilobases (kb) in length (e.g., about 10 bp, 50 bp, 100 bp, 500 bp, 1 kb, 5 kb, 10 kb, 50 kb, 100 kb, 500 kb, or 1000 kb in length).

A genetic variation is sometimes a deletion. In certain embodiments a deletion is a mutation (e.g., a genetic aberration) in which a part of a chromosome or a sequence of DNA is missing. A deletion is often the loss of genetic material. Any number of nucleotides can be deleted. A deletion can comprise the deletion of one or more entire chromosomes, a segment of a chromosome, an allele, a gene, an intron, an exon, any non-coding region, any coding region, a segment thereof or combination thereof. A deletion can comprise a microdeletion. A deletion can comprise the deletion of a single base.

A genetic variation is sometimes a genetic duplication. In certain embodiments a duplication is a mutation (e.g., a genetic aberration) in which a part of a chromosome or a sequence of DNA is copied and inserted back into the genome. In certain embodiments a genetic duplication (i.e. duplication) is any duplication of a region of DNA. In some embodiments a duplication is a nucleic acid sequence that is repeated, often in tandem, within a genome or chromosome. In some embodiments a duplication can comprise a copy of one or more entire chromosomes, a segment of a chromosome, an allele, a gene, an intron, an exon, any non-coding region, any coding region, segment thereof or combination thereof. A duplication can comprise a microduplication. A duplication sometimes comprises one or more copies of a duplicated nucleic acid. A duplication sometimes is characterized as a genetic region repeated one or more times (e.g., repeated 1, 2, 3, 4, 5, 6, 7, 8, 9 or 10 times). Duplications can range from small regions (thousands of base pairs) to whole chromosomes in some instances. Duplications frequently occur as the result of an error in homologous recombination or due to a retrotransposon event. Duplications have been associated with certain types of proliferative diseases. Duplications can be characterized using genomic microarrays or comparative genetic hybridization (CGH).

A genetic variation is sometimes an insertion. An insertion is sometimes the addition of one or more nucleotide base pairs into a nucleic acid sequence. An insertion is sometimes a microinsertion. In certain embodiments an insertion comprises the addition of a segment of a chromosome into a genome, chromosome, or segment thereof. In certain embodiments an insertion comprises the addition of an allele, a gene, an intron, an exon, any non-coding region, any coding region, segment thereof or combination thereof into a genome or segment thereof. In certain embodiments an insertion comprises the addition (i.e., insertion) of nucleic acid of unknown origin into a genome, chromosome, or segment thereof. In certain embodiments an insertion comprises the addition (i.e. insertion) of a single base.

A genetic variation sometimes includes copy number variations, i.e., variations in the number of copies of a nucleic acid sequence present in a test sample in comparison with the copy number of the nucleic acid sequence present in a reference sample. In certain embodiments, the nucleic acid sequence is 1 kb or larger. In some cases, the nucleic acid sequence is a whole chromosome or significant portion thereof. A copy number variant may refer to the sequence of nucleic acid in which copy-number differences are found by comparison of a nucleic acid sequence of interest in test sample with an expected level of the nucleic acid sequence of interest. For example, the level of the nucleic acid sequence of interest in the test sample is compared to that present in a qualified sample. Copy number variants/variations may include deletions, including microdeletions, insertions, including microinsertions, duplications, multiplications, and translocations. CNVs encompass chromosomal aneuploidies and partial aneuploidies.

As used herein, the term “array” may refer to a sequence of given size in the genome. In some examples, an array may comprise the total length of a VNTR. In some examples, an array may include all of the repeat copies of a VNTR. In some examples, an array may further comprise another target region.

As used herein, the term “consensus pattern motif (logo)” refers to the consensus sequence of the VNTR pattern describing the frequency at which different bases occur at each position.

As used herein, the term “copy number” refers to the number of times (e.g., 0, 1, 1.5, 2, 3.5, 5, etc.) the repeat unit is repeated for a given VNTR. The change in copy number for a VNTR can be represented as the difference in copy number relative to the reference (e.g., −1, 0, +1, +2, etc.).

As used herein, the term “fragment size” refers to the length of the original nucleic acid sequence used to generate paired-end reads, calculated based on where those reads are mapped.

As used herein, the term “indels” refers to small insertions or deletions less than 50 base pairs in length in a nucleic acid sequence.

As used herein, the term “paired-end reads” or “paired end reads” refers to paired reads generated from sequencing the forward and reverse ends of a larger nucleic acid fragment. In some examples, the forward and reverse ends of a larger nucleic acid fragment may share the same name. The paired-end reads may be generated from paired end sequencing that obtains one read from each end of a nucleic acid fragment.

As used herein, the term “pattern” refers to the sequence of a repeat unit of the tandem repeat.

As used herein, the term “mate” or “mate of a read” refers to the pair of the read in question; i.e., the other read generated from the same nucleic acid fragment.

As used herein, the term “repeat unit” refers to the sequence of a single copy that is repeated multiple times in a VNTR.

As used herein, the term “single nucleotide variants” or “SNVs” refers to single base substitutions in a nucleic acid sequence.

As used herein, the term “small variant event” refers to a collection of adjacent SNVs or indels that occurs in the same haplotype of the VNTR array within a maximal distance of each other (for example, a maximal distance of 10 base-pairs).

As used herein, the term “spanning fragment” refers to a read fragment that spans the length of the entire VNTR array such that the left and right paired-end reads are on the left and right flanks of the VNTR.

As used herein, the term “structural variation” or “SV” refers to a large nucleic acid variant greater than 50 base pairs corresponding to either a duplication, deletion, insertion, inversion, or translocation.

As used herein, the term “tandem repeat” or “TR” refers to a nucleic acid sequence with a repeat unit of at least 10 base pairs, where the repeat unit is repeated at least 1.6 times with a similarity score of at least 1.7, consistent with the definitions in “Benson, Gary. ‘Tandem repeats finder: a program to analyze DNA sequences.’ Nucleic acids research 27.2 (1999): 573-580, the disclosures of which are incorporated herein by reference in their entirety.

As used herein, the term “variable number tandem repeat” or “VNTR” refers to a tandem repeat that has been observed to vary in the number of copies (e.g., duplicated copies or deleted copies) in the population of a species.

As used herein, the term “VNTR array” refers to the sequence covering the entire length of a VNTR. The VNTR array includes all of the copies of the repeat units.

In some cases, two haplotypes of a VNTR may comprise different numbers of copies of the repeat unit. In some cases, two haplotypes of the VNTR may comprise an identical number of copies of the repeat unit. The repeat units in each of the two haplotypes can include differentiating bases. A sequence of the repeat unit of one of the two haplotypes and a sequence of the repeat unit of the other one of the two haplotypes can be different at one or more differentiating positions; these sequences can have (or can have at least) 70%, 75%, 80%, 85%, 90%, 95%, 99%, or more, sequence identity. A sequence of the repeat unit of one of the two haplotypes and a sequence of the repeat unit of the other one of the two haplotypes can be identical in some examples.

Each haplotype of a VNTR can comprise a plurality of copies of a repeat unit. The repeat unit can be (or be at least or be more than) 6 bps, 7 bps, 8 bps, 9 bps, 10 bps, 11 bps, 12 bps, 13 bps, 14 bps, 15 bps, 16 bps, 17 bps, 18 bps, 19 bps, 20 bps, or more in length. The number of the plurality of copies can be (or be at least or be more than) 1.6, or more. The pathogenic copy number can be equal to, more than, or less than, the copy number in the reference sequence.

Two copies of a repeat unit of a haplotype can include differentiating bases. For example, sequences of two copies of the repeat unit of a haplotype can be different at one or more differentiating positions (e.g., 2, 3, 4, 5, 10, 20, or more, positions). The sequences of the two copies of the repeat unit of a haplotype may have (or may have at least) 70%, 75%, 80%, 85%, 90%, 95%, 99%, or more, sequence identity. Sequences of two copies of the repeat unit of a haplotype can be identical in some examples.

### ADDITIONAL NOTES

The embodiments described herein are exemplary. Modifications, rearrangements, substitute processes, etc. may be made to these embodiments and still be encompassed within the teachings set forth herein. One or more of the steps, processes, or methods described herein may be carried out by one or more processing and/or digital devices, suitably programmed.

The various illustrative imaging or data processing techniques described in connection with the embodiments disclosed herein can be implemented as electronic hardware, computer software, or combinations of both. To illustrate this interchangeability of hardware and software, various illustrative components, blocks, modules, and steps have been described above generally in terms of their functionality. Whether such functionality is implemented as hardware or software depends upon the particular application and design constraints imposed on the overall system. The described functionality can be implemented in varying ways for each particular application, but such implementation decisions should not be interpreted as causing a departure from the scope of the disclosure.

The various illustrative detection systems described in connection with the embodiments disclosed herein can be implemented or performed by a machine, such as a processor configured with specific instructions, a digital signal processor (DSP), an application specific integrated circuit (ASIC), a field programmable gate array (FPGA) or other programmable logic device, discrete gate or transistor logic, discrete hardware components, or any combination thereof designed to perform the functions described herein. A processor can be a microprocessor, but in the alternative, the processor can be a controller, microcontroller, or state machine, combinations of the same, or the like. A processor can also be implemented as a combination of computing devices, e.g., a combination of a DSP and a microprocessor, a plurality of microprocessors, one or more microprocessors in conjunction with a DSP core, or any other such configuration. For example, systems described herein may be implemented using a discrete memory chip, a portion of memory in a microprocessor, flash, EPROM, or other types of memory.

The elements of a method, process, or algorithm described in connection with the embodiments disclosed herein can be embodied directly in hardware, in a software module executed by a processor, or in a combination of the two. A software module can reside in RAM memory, flash memory, ROM memory, EPROM memory, EEPROM memory, registers, hard disk, a removable disk, a CD-ROM, or any other form of computer-readable storage medium known in the art. An exemplary storage medium can be coupled to the processor such that the processor can read information from, and write information to, the storage medium. In the alternative, the storage medium can be integral to the processor. The processor and the storage medium can reside in an ASIC. A software module can comprise computer-executable instructions which cause a hardware processor to execute the computer-executable instructions.

Conditional language used herein, such as, among others, “can,” “might,” “may,” “e.g.,” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments or that one or more embodiments necessarily include logic for deciding, with or without author input or prompting, whether these features, elements and/or states are included or are to be performed in any particular embodiment. The terms “comprising,” “including,” “having,” “involving,” and the like are synonymous and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list.

Disjunctive language such as the phrase “at least one of X, Y or Z,” unless specifically stated otherwise, is otherwise understood with the context as used in general to present that an item, term, etc., may be either X, Y or Z, or any combination thereof (e.g., X, Y and/or Z). Thus, such disjunctive language is not generally intended to, and should not, imply that certain embodiments require at least one of X, at least one of Y or at least one of Z to each be present.

The terms “about” or “approximate” and the like are synonymous and are used to indicate that the value modified by the term has an understood range associated with it, where the range can be ±20%, ±15%, ±10%, ±5%, or ±1%. The term “substantially” is used to indicate that a result (e.g., measurement value) is close to a targeted value, where close can mean, for example, the result is within 80% of the value, within 90% of the value, within 95% of the value, or within 99% of the value.

Unless otherwise explicitly stated, articles such as “a” or “an” should generally be interpreted to include one or more described items. Accordingly, phrases such as “a device configured to” or “a device to” are intended to include one or more recited devices. Such one or more recited devices can also be collectively configured to carry out the stated recitations. For example, “a processor to carry out recitations A, B and C” can include a first processor configured to carry out recitation A working in conjunction with a second processor configured to carry out recitations B and C.

While the above detailed description has shown, described, and pointed out novel features as applied to illustrative embodiments, it will be understood that various omissions, substitutions, and changes in the form and details of the devices or algorithms illustrated can be made without departing from the spirit of the disclosure. As will be recognized, certain embodiments described herein can be embodied within a form that does not provide all of the features and benefits set forth herein, as some features can be used or practiced separately from others. All changes which come within the meaning and range of equivalency of the claims are to be embraced within their scope.

It should be appreciated that all combinations of the foregoing concepts (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein.

1. A method of estimating a copy number of repeat units in a target genomic region in a nucleic acid sample from a subject, the target genomic region corresponding to a tandem repeat locus, the method comprising:
obtaining known copy numbers of repeat units in predetermined invariant tandem repeat loci;
training a machine learning model to learn the relationship between:
(a) the obtained copy numbers of repeat units in the predetermined invariant tandem repeat loci, and
(b) specific features of the predetermined loci and (b-ii) specific features of training genomic regions in the nucleic acid sample from the subject that correspond to the predetermined loci;
obtaining paired-end sequence reads of the target genomic region; and
estimating the copy number of repeat units in the target genomic region by applying the trained machine learning model to specific features of the tandem repeat locus and specific features of the target genomic region.

2. The method of claim 1, wherein the specific features of the tandem repeat locus comprise: a size of the tandem repeat locus, a size of a repeat unit in the tandem repeat locus, or a GC content of the tandem repeat locus.

3. The method of claim 1, wherein the specific features of the target genomic region comprise: a number of paired-end sequence reads resulting from the target genomic region that are spanning the tandem repeat locus, a number of paired-end sequence reads resulting from the target genomic region that are overlapping the tandem repeat locus, a number of paired-end sequence reads resulting from the target genomic region that are overlapping the start and end positions of the tandem repeat locus, or a number of paired-end sequence reads resulting from the target genomic region that are contained inside the tandem repeat locus.

4. The method of claim 1, wherein obtaining the paired-end sequence reads of the target genomic region comprises: performing paired-end sequencing of the nucleic acid sample from the subject, aligning the paired-end sequence reads to a reference genomic sequence for the species of the subject, and obtaining the paired-end sequence reads that align to the tandem repeat locus on the reference genomic sequence.

5. The method of claim 1, wherein the known copy numbers of repeat units in the predetermined invariant tandem repeat loci are obtained from a database or a reference genomic sequence.

6. The method of claim 1, wherein each of the predetermined loci is invariant among subjects of the same species.

7. The method of claim 1, wherein the machine learning model is a XGBoost model, a regression model, a random forest model or a decision tree model.

8. The method of claim 1, wherein training the machine learning model comprises:
inputting the obtained copy numbers of repeat units in the predetermined invariant tandem repeat loci, the specific features of the predetermined loci, and the specific features of the training genomic regions corresponding to the predetermined loci to a machine learning module;
optimizing the machine learning model using the machine learning module and said inputs; and
outputting the trained machine learning model wherein model parameters characterizing the relationship between the obtained copy numbers and the specific features of the predetermined loci and the specific features of the training genomic regions are optimized.

9. The method of claim 8, wherein optimizing the machine learning model comprises:
estimating predicted copy numbers of repeat units in the predetermined invariant tandem repeat loci by applying the machine learning model to the specific features of the predetermined loci and the specific features of the training genomic regions corresponding to the predetermined loci;
comparing said predicted copy numbers with the obtained copy numbers of repeat units in the predetermined invariant tandem repeat loci; and
updating the machine learning model based in part on said comparison.

10. The method of claim 1, wherein the specific features of the predetermined loci comprise: a size of the predetermined loci, a size of a repeat unit in the predetermined loci, or a GC content of the predetermined loci.

11. The method of claim 1, wherein the specific features of the training genomic regions corresponding to the predetermined loci comprise: a number of paired-end sequence reads resulting from the training genomic regions that are spanning the predetermined loci, a number of paired-end sequence reads resulting from the training genomic regions that are overlapping the predetermined loci, a number of paired-end sequence reads resulting from the training genomic regions that are overlapping the start and end positions of the predetermined loci, or a number of paired-end sequence reads resulting from the training genomic regions that are contained inside the predetermined loci.

12. The method of claim 1, further comprising reporting a confidence level for said estimation, wherein the confidence level is determined based in part on control genomic regions in the nucleic acid sample that correspond to control loci comprising invariant tandem repeats.

13. (canceled)

14. The method of claim 12, wherein determining the confidence level comprises:
estimating predicted copy numbers of repeat units in the invariant tandem repeats of the control loci by applying the machine learning model to specific features of the control loci and specific features of the control genomic regions;
comparing said predicted copy numbers with known copy numbers of repeat units in the invariant tandem repeats of the control loci; and
determining the confidence level based on said comparison.

15. The method of claim 14, wherein the known copy numbers of repeat units in the invariant tandem repeats of the control loci are obtained from a database or a reference genomic sequence.

16. The method of claim 14, wherein the specific features of the control loci comprise: a size of the control loci, a size of a repeat unit in the control loci, or a GC content of the control loci.

17. The method of claim 14, wherein the specific features of the control genomic regions comprise: a number of paired-end sequence reads resulting from the control genomic regions that are spanning the control loci, a number of paired-end sequence reads resulting from the control genomic regions that are overlapping the control loci, a number of paired-end sequence reads resulting from the control genomic regions that are overlapping the start and end positions of the control loci, or a number of paired-end sequence reads resulting from the control genomic regions that are contained inside the control loci.

18. The method of claim 1, wherein the nucleic acid sample comprises both paternal and maternal nucleic acids for the subject.

19. The method of claim 1, wherein the nucleic acid sample is extracted from cells, a cell-free DNA sample, an amniotic fluid, a blood sample, a biopsy sample, or any combination thereof, of the subject.

20.-23. (canceled)

24. The method of claim 1, wherein each paired-end sequence read is about 100 base pairs to about 500 base pairs in length.

25. The method of claim 1, wherein the paired-end sequence reads for the nucleic acid sample is generated by whole genome sequencing (WGS).

26.-33. (canceled)
